(19) United States
(12) Patent Application Publication
(10) Pub. No.: US 2023/0054250 A1
(43) Pub. Date: Feb. 23, 2023

(54) NON-AQUEOUS SUSTAINED RELEASE DRUG DELIVERY SYSTEM

(71) Applicant: SHANGHAI INSTITUTE OF MATERIA MEDICA, CHINESE ACADEMY OF SCIENCES, Shanghai (CN)

(72) Inventors: Yong GAN, Shanghai (CN); Shiyan GUO, Shanghai (CN); Yinyin SUN, Shanghai (CN)

(21) Appl. No.: 17/792,499

(22) PCT Filed: Jan. 14, 2021
(86) PCT No.: PCT/CN2021/071648

§ 371 Date: Sep. 7, 2022

(30) Foreign Application Priority Data
Jan. 14, 2020 (CN) 202010037081.8

(51) Int. Cl. A61K 9/127; A61K 31/167; A61K 31/277; A61K 31/5415; A61K 31/4748; A61K 31/445; A61K 31/138; A61K 31/475; A61K 31/4365; A61K 31/245; A61K 31/366; A61K 31/4422; A61K 47/10; A61K 47/24; A61K 47/14; A61K 47/44; A61K 47/28; A61K 9/00

(52) U.S. Cl. CPC A61K 9/127; A61K 9/0019; A61K 31/138; A61K 31/167; A61K 31/245; A61K 31/277; A61K 31/366; A61K 31/445; A61K 31/475; A61K 31/4365; A61K 31/4422; A61K 31/4748; A61K 31/5415; A61K 47/10; A61K 47/14; A61K 47/24; A61K 47/28; A61K 47/44

(57) ABSTRACT

A non-aqueous sustained release drug delivery system, which contains, based on the total weight of the sustained release drug delivery system, about 0.1-20% of an active agent, about 0.5-50% of a drug solvent, about 1-98% of a drug sustained release agent, about 0.1-85% of a drug solubilizer, about 0.1-10% of an efficacy enhancer, about 0-1% of an antioxidant, and about 0-8% of an acid-base regulator. The non-aqueous sustained release drug delivery system can yield an improved sustained release effect, improve bioavailability, and enhance the therapeutic effect.

### TECHNICAL FIELD

The invention belongs to the field of pharmacy, and mainly relates to a non-aqueous sustained release drug delivery system, which can provide improved sustained release effect, increase bioavailability and enhance efficacy. Compared with conventional sustained release preparations, the composition of the present invention can improve effective bioavailability and drug utilization, reduce drug dosage under the same drug effect, and reduce the toxicity risk caused by accumulation of ineffective drugs. The non-aqueous sustained release drug delivery system can control and adjust the release rate of active agents in vivo, providing beneficial value for on-demand selection during drug development. The obtained drug delivery system has good in vivo safety, controllable sustained release effect and high bioavailability, thus having a good application prospect.

### BACKGROUND

Many active agents (such as antibiotics, antiseptics, corticosteroids, antineoplastic drugs, and local anesthetics, etc.) can be applied to the skin or mucous membranes by topical application or injection, and then act locally or systemically. Topical delivery can be accomplished by using ointments, creams, emulsions, solutions, suspensions, and the like. Injections for delivery of active agents include solutions, suspensions, emulsions, and the like. Although these preparations have been widely used, they usually have a short duration and require multiple administrations, which may lead to poor patient compliance with high treatment costs, and also to a large peak-to-valley ratio, resulting in a series of toxic and side effects. Therefore, it is necessary to develop an injectable drug sustained release delivery system, which can release the drug slowly to achieve a stable blood drug concentration and reduce the peak-to-valley value to reduce the toxicity, increase the efficacy, increase the patient’s medication compliance and meet clinical needs.

In recent years, dosage forms with a certain sustained release effect have been developed, including liposomes, microcapsules, microspheres, in situ gels, nanocrystal suspensions, etc. These preparations have advantages in that the active agent can be gradually released for a long time, without repeated dosing.

At present, one example of the marketed liposome preparations is the bupivacaine multivesicular liposome (trade name: Exparel) using Depofoam technology, which can achieve analgesic effect for about 24 hours. Although it has better application advantages than traditional preparations, there are still some shortcomings. First, it has a complex formulation and requires the presence of neutral lipids, otherwise unilamellar liposomes or multilamellar liposomes will be formed. Secondly, the preparation process is complicated, and two emulsification processes are needed for the preparation, which is difficult for industrial production. Finally, it has poor stability, because there is a difference in drug concentration between the internal and external water phases of the product, which is prone to leakage of small molecule drugs, and thus it needs to be stored at low temperature. Moreover, this product is a suspension, and vesicle rupture, sedimentation and aggregation may occur during storage, which is unbeneficial for storage and transportation. Thus, its development is limited.

As for microsphere preparations, one of the representative varieties is risperidone sustained release microsphere injection under the trade name of Risperdal Consta, which is developed by Johnson & Johnson, US, marketed sequentially in US and Europe in 2003 and then in China in 2006, and can release for 2 weeks. Luye Pharma developed its own risperidone sustained release microsphere intramuscular injection preparation (LY3004) for the treatment of schizophrenia, for which 3 pivotal Phase I clinical trials have been completed and it only needs to be injected every 2 weeks and is easy for use. Although the microspheres have a good sustained release effect, they have complicated preparation process, low drug loading and high production cost, and the degradation products, lactic acid and glycolic acid, can cause pH change at the injection site, thereby causing adverse reactions at the injection site. These shortcomings limit the application of the microspheres in the field of long-acting preparations.

Representative preparations of the marketed in situ gel preparations are Atridox and Eligard using Atrigel Delivery system, and both of them use polylactic acid-glycolic acid copolymer (PLGA) as the sustained release material. The sustained release time can be adjusted according to the type of PLGA selected, and the release time may be 7 days, 1 month, 3 months, 4 months, 6 months, etc. Although the system has a relatively controllable sustained release effect, it uses N-methylpyrrolidone (NMP) as a solvent, and the content of the solvent is at least 50%, which will cause strong irritation at the injection site with poor patient tolerance. In addition, such preparations are generally pre-filled two-bottle preparations, thus it requires medical staff to mix evenly before use, which operation may lead to differences during mixing and inaccuracy of injection dosages, causing inconvenience in clinical use.

One representative variety of nanocrystalline suspensions is Paliperidone Palmitate Injection under the trade name of Xeplion, which was developed by Janssen-Cilag International Company, and approved by the European Commission in March 2011 for intramuscular injection once a month for the treatment of psychosis schizophrenia. This variety is prepared by Elan’s unique “Nano Crystallization Technology”, and the drug effect lasts up to 1 month. However, in development of a nanosuspension, it is often necessary to consider the possibility of particle growth during storage and its impact on agglomeration, precipitation, resuspension, and dissolution rates, imposing challenges in development.

CN108159055A discloses a long-acting drug delivery system for treating breast cancer, which uses fulvestrant as the main drug, phospholipids or a mixture of phospholipids and oil as the sustained release material, and ethanol or benzyl alcohol as the drug solvent. CN102131483A discloses a controllable drug delivery non-polymer composition, which is composed of a hydrophobic non-polymer carrier material, a solvent and an ionic complex formed by a biologically active substance and an amphiphilic molecule, and certain additives such as cephalin and lecithin etc., may be added. Although the systems in the above studies have a certain sustained release effect, they all have the disadvantage of low drug effective availability.

Although the above systems have certain application value, they all have disadvantages such as complex production process, low drug effective availability, high cost, high safety risk, and poor patient tolerance, etc.

Therefore, it is very important to develop a sustained release drug delivery system with simple preparation, low cost, high drug effective availability, good safety and good patient tolerance.

### SUMMARY OF THE INVENTION

Based on the above needs, the inventors of the present application have developed a non-aqueous sustained release drug delivery system, which can significantly reduce the plasma peak concentration of the drug, with a controllable release rate and obvious sustained release effect. Further, in some embodiments, the non-aqueous sustained release drug delivery system of the present invention can improve the bioavailability of the drug, reduce drug dose under the same efficacy. In some embodiments, the non-aqueous sustained release drug delivery system of the present invention can improve the effective bioavailability of the drug, avoid the retention of ineffective drugs, and reduce the toxic reaction caused by drug accumulation, thus having the advantages of high safety and good tolerance. These have not been reported in this field yet.

In the invention, the effective therapeutic dosage is calculated according to the following formula,

$$R = A / {T}_{t}$$

Where A is the dose of the drug (sometimes referred to as the active agent in the invention), Tₜ is the effective treatment time of the drug, that is, the in vivo duration of action of the drug, which can be measured through the pharmacodynamic experiment. The smaller the R value, the smaller the drug dose consumed to achieve the same duration of action, that is, the longer the effective treatment time of the drug under the same dose.

An object of the invention is to overcome the defects in the prior art and provide a non-aqueous sustained release drug delivery system with wide application range and high safety. In some embodiments, the non-aqueous sustained release drug delivery system of the invention has significantly reduced Cₘₐₓ compared with the existing ordinary injection at the same dosage. In some embodiments, the non-aqueous sustained release drug delivery system of the invention has increased in vivo exposure of the drug, improved bioavailability and prolonged effective treatment time compared with the existing conventional sustained release system. In some embodiments, the non-aqueous sustained release drug delivery system of the invention can significantly reduce the drug dosage, improve the drug availability and reduce the risk of adverse reactions caused by drug accumulation under the same duration of action, compared with the existing conventional sustained release preparations. In some embodiments, the non-aqueous sustained release drug delivery system of the invention can controllably adjust the release rate of the drug, and the duration of action can reach at least 12h, which can meet different clinical needs.

Another object of the invention is to provide a method of preparing the non-aqueous sustained release drug delivery system.

In one aspect, the invention provides a non-aqueous sustained release drug delivery system, which comprises an active agent, a drug solvent, a drug sustained release agent, a drug solubilizer, an efficacy enhancer, an optional antioxidant and an optional acid-base regulator.

Preferably, the sustained release drug delivery system of the invention comprises: based on the total weight of the sustained release drug delivery system,
0.1% to 20%, preferably 0.5% to 15%, more preferably 1% to 10% of the active agent;
0.5% to 50%, preferably 2% to 40%, more preferably 5% to 35% of the drug solvent;
1% to 98%, preferably 5% to 90%, more preferably 10% to 82% of the drug sustained release agent;
1% to 85%, preferably 5% to 80%, more preferably 10% to 65% of the drug solubilizer;
0.1% to 10%, preferably 1% to 10%, more preferably 2% to 8% of the efficacy enhancer;
0% to 1%, preferably 0% to 0.5%, more preferably 0% to 0.3% of the antioxidant;
0% to 8%, preferably 0% to 5%, more preferably 0% to 2% of the acid-base regulator.

The active agent in the drug delivery system of the invention is any compound that produces beneficial or useful effects or a mixture thereof. Suitable active agents include pharmaceutical active agents with local or systemic effects, which can be applied to patients by local or intralesional application (including, for example, application to injured skin, laceration, puncture wound, etc., and application to surgical incision) or by injection (such as subcutaneous, intradermal, intramuscular, intraocular, or intra-articular injection).

The active agents can be water-soluble molecules, fat soluble molecules or amphiphilic molecules, including but not limited to: anticancer drugs, anti-inflammatory drugs, anti-infective drugs, painkillers, hormones, anti-diabetic drugs, anti-hypertension drugs, anti-AIDS drugs, immune enhancers, anti-viral drugs, cardiotonics, anti-obesity drugs, bone metabolism regulators, antiepileptics, anticonvulsants, antidepressants, antipsychotics, anti-Parkinson’s disease drugs, urinary tract drugs, contraceptives, anti-osteoporosis drugs, protein assimilation agents, smoking cessation aids and cell adhesion promoters.

Examples of these active agents include, but are not limited to, anti infectives (including antibiotics, anti-viral agents, fungicides, scabies killing agents or lice killing agents), preservatives (such as benzalkonium chloride, benzethonium chloride, chlorhexidine gluconate, sulfamylone acetate, methylbenzalkonium chloride, furacillin, metaphen, etc.), steroids (such as estrogens, progesterones, androgens, adrenocortical sterols, etc.), therapeutic peptides (such as insulin, erythropoietin, morphogenetic proteins such as bone morphogenetic protein, etc.), analgesics and anti-inflammatory agents (such as aspirin, ibuprofen, naproxen, ketorolac, lappaconitine, bulleyaconitine A, COX-1 inhibitors, COX-2 inhibitors, etc.), cancer chemotherapeutic agents (such as mechlorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen, fulvestrant, etc.), narcotic drugs (such as morphine, pethidine, codeine, etc.), local anesthetics (such as amide or ester local anesthetics, such as bupivacaine, dibucaine, mepivacaine, procaine, lidocaine, tetracaine, ropivacaine, etc.), antiemetics (such as ondansetron, granisetron, tropisetron, metoclopramide, domperidone, scopolamine, etc.), antiangiogenic agents (such as combretastatin, anti-VEGF, etc.), cardiovascular drugs (such as clopidogrel), antihistamines (such as diphenhydramine, chlorpheniramine, promethazine, etc.), antihypertensive drugs (such as hydrochlorothiazide, amiloride, furosemide, clonidine, rimenidine, reserpine, propranolol, captopril, perindopril, losartan, candesartan, remikiren, nifedipine, L-amlodipine, diltiazem, verapamil, atenolol, sotalol, metoprolol), lipid lowering drugs (such as simvastatin, lovastatin, atorvastatin, rosuvastatin, fenofibrate, bezafibrate, ezetimibe, etc.), nervous system drugs (such as nimodipine, carbamazepine, fluoxetine, etc.), cholinesterase inhibitors (such as donepezil, huperzine A, etc.), polysaccharides, vaccines, antigens, DNA and other polynucleotides, antisense oligonucleotides, etc. The invention can also be applied to other locally acting active agents, such as astringents, antiperspirants, irritants, reddening agents, foaming agents, hardeners, corrosives, caustic agents, horny softeners, sunscreens, and various skin disease agents. Prodrugs of active agents are included within the scope of the present invention.

The drug solvent in the drug delivery system of the invention is a single organic solvent or a mixture of multiple organic solvents. The organic solvent can be one or more selected from the group consisting of benzyl alcohol, ethanol, glycerol, isopropanol, liquid polyethylene glycol, polyethylene glycol monomethyl ether, glycerol monoacetate, diethylene glycol monoethyl ether, ethyl lactate, tetrahydrofuran polyethylene glycol ether, benzyl benzoate, dimethyl acetamide, N-methyl pyrrolidone, 2-pyrrolidone, propylene glycol, methyl acetate, ethyl acetate, propylene glycol diethyl ester, diethyl malonate, glyceryl triacetate, dimethylformamide, dimethyl sulfoxide, caprolactam, triethyl citrate and propylene carbonate, but is not limited thereto.

The drug sustained release agent in the drug delivery system of the invention is one or more selected from the group consisting of biodegradable polymers and pharmaceutical oils; preferably one or more selected from the group consisting of pharmaceutical oils.

Specifically, the biodegradable polymers can be one or more selected from the group consisting of polylactic acid (PLA), polylactic acid-glycolic acid copolymer (PLGA), polyorthoesters, sucrose acetate iso-butyrate, glycerin fatty acid ester, polyethylene glycol (PEG) modified(PEGylated) PLA/PLGA, PLGA-PEG-PLGA copolymer, triethylene glycol poly(orthoester) polymer, chitosan, water-soluble carboxymethyl chitosan, fibroin, poly-β-hydroxybutyrate valerate, polylactide/lactide-polyethylene glycol copolymer and/or blend thereof, polycaprolactone-polyethylene glycol copolymer, blend of poly β-hydroxybutyrate and polyethylene glycol, and blend of polylactic acid/hydroxyacetic acid.

Specifically, the pharmaceutical oil can be one or more selected from the group consisting of castor oil, sesame oil, soybean oil, sunflower seed oil, peanut oil, corn oil, rapeseed oil, olive oil, cotton seed oil or other natural vegetable oils, semi-natural oils artificially modified from natural vegetable oils (such as hydrogenated castor oil), purified oils and corresponding derivatives; and synthetic oils, mainly including medium chain (a carbon chain length of C₆-C₁₂) triglycerides (e.g. one of caprylic triglyceride, capric triglyceride or a mixture of both), long chain (a carbon chain length of C₁₃-C₂₄) triglycerides, triacetin or other corresponding derivatives, ethyl oleate.

Among them, the drug sustained release agent is preferably one or more selected from the group consisting of castor oil, sesame oil, ethyl oleate, soybean oil, medium chain triglyceride and peanut oil, and more preferably one or more selected from the group consisting of castor oil, soybean oil and sesame oil.

The drug solubilizer in the drug delivery system of the invention is one or more selected from the group consisting of pharmaceutical surfactants. Specifically, the pharmaceutical surfactant can be one or more selected from the group consisting of pharmaceutical phospholipids, polyoxyl 15 hydroxystearate, polysorbate, polyoxyethylene castor oil, poloxamer, polyoxyethylene fatty acid ester, phosphatidylcholine (such as DEPC or DOPC or a composition thereof), phosphatidylglycerol (such as DPPG), polyethylene glycol, polyethylene glycol monomethyl ether and gelatin, preferably one or more selected from the group consisting of pharmaceutical phospholipids. Specifically, the pharmaceutical phospholipid can be one or more selected from the group consisting of natural phospholipids, semi-synthetic phospholipids, and synthetic phospholipids. The natural phospholipids include, but are not limited to, egg yolk lecithin, soybean lecithin or combinations thereof. The semi-synthetic phospholipids include, but are not limited to, hydrogenated egg yolk lecithin, hydrogenated soybean phospholipids or combinations thereof. The synthetic phospholipids include, but are not limited to, dipalmitoyl phosphatidylethanolamine, dipalmitoyl phosphatidic acid, dipalmitoyl- phosphatidylglycerole, dioleoylphosphatidylethanolamine, dipalmitoylphosphatidylcholine, distearoylphosphatidylcholine, dimyristoyl phosphatidyl choline, or combinations thereof. The pharmaceutical phospholipid in the drug delivery system of the invention is preferably a natural phospholipid, i.e., a non-synthetic phosphatidylcholine (PC) or a pharmaceutically acceptable salt thereof, including, but not limited to, egg yolk lecithin, soybean lecithin or a combination thereof.

The efficacy enhancer in the drug delivery system of the invention is one or more selected from the group consisting of ω-3 fatty acids and metabolites thereof, substances rich in ω-3 fatty acids or metabolites thereof, glucocorticoids, phosphodiesterase-4 inhibitors, etc.

The ω-3 fatty acids and metabolites thereof mainly refer to ω-3 polyunsaturated fatty acids and metabolites thereof, such as α-linolenic acid and its metabolites eicosapentaenoic acid (EPA) and docosahexaenoic acid (DHA). The α-linolenic acid involved is the α-linolenic acid (ALA) of plant origin.

The substances rich in ω-3 fatty acids or metabolites thereof include substances rich in α-linolenic acid, substances rich in α-linolenic acid metabolites (such as EPA, DHA, etc.). The substances rich in α-linolenic acid can be linseed oil, perilla seed oil, walnut oil, argan oil and other vegetable oils or combinations thereof. The substances rich in α-linolenic acid metabolites can be fish oil, laver oil, algal oil or combinations thereof.

In some embodiments, the substance rich in ω-3 fatty acids or metabolites thereof is preferably one or more selected from the group consisting of substances rich in ω-3 fatty acids or metabolites thereof with eicosapentaenoic acid content of not less than 15% and docosahexaenoic acid content of not less than 10%, more preferably one or more selected from the group consisting of substances rich in ω-3 fatty acids or metabolites thereof with eicosapentaenoic acid content of not less than 20% and docosahexaenoic acid content of not less than 10%, and more particularly preferably one or more selected from the group consisting of fish oil and laver oil.

The glucocorticoids include, but are not limited to, prednisone, methylprednisone, betamethasone, beclomethasone propionate, prednisolone, hydrocortisone, dexamethasone or combinations thereof.

The phosphodiesterase-4 inhibitors include, but are not limited to, roflumilast, rolipram, pentoxifylline or combinations thereof.

The efficacy enhancer in the drug delivery system of the invention is preferably a substance rich in ω-fatty acids or metabolites thereof.

The antioxidant in the drug delivery system of the invention is a single antioxidant or a mixture of multiple antioxidants. The antioxidant may be one or more selected from the group consisting of cysteine, α-tocopherol, α-tocopherol acetate, N-acetyl-L-cysteine, butylated hydroxyanisole, dibutylated hydroxytoluene, propyl gallate, tert-butyl hydroquinone, lipoic acid, tea polyphenols, L-ascorbyl palmitate, glutathione; preferably one or more selected from the group consisting of α-tocopherol and L-ascorbyl palmitate.

The acid-base regulator in the drug delivery system of the invention is one or more selected from the group consisting of arginine, lysine, histidine, glycine, tromethamine, diethanolamine, ethylenediamine, meglumine, hydrochloric acid, acetic acid, anhydrous citric acid, ascorbic acid, lactic acid, tartaric acid, methanesulfonic acid, methionine, sodium hydroxide and triethanolamine.

In another aspect, the invention provides a method of preparing the non-aqueous sustained release drug delivery system. None of the stages in the process of preparing the non-aqueous sustained release drug delivery system of the invention is exposed to an aqueous phase. The composition does not contain water except for residual moisture that may exist in the raw materials for preparing the composition.

In one embodiment, the preparation method can be one of the following methods, but is not limited to.

Method 1, it comprises the following steps:
(1) adding an active agent into a drug solvent and dispersing the active agent to obtain a dispersion of the active agent;
(2) mixing a drug solubilizer and a drug sustained release agent well;
(3) adding the active agent dispersion and an efficacy enhancer into the liquid obtained in step (2), mixing them well and filtering the resultant to obtain the non-aqueous sustained release drug delivery system.

Optionally, the antioxidant and the acid-base regulator can be added in any step of the above process.

Method 2, it comprises the following steps:
(1) adding an active agent into a drug solvent and dispersing the active agent well to obtain a dispersion of the active agent;
(2) adding a drug solubilizer into the active agent dispersion obtained in step (1) and mixing them well;
(3) adding a drug sustained release agent and an efficacy enhancer into the liquid obtained in step (2), mixing them well and filtering the resultant to obtain the non-aqueous sustained release drug delivery system.

Optionally, the antioxidant and acid-base regulator can be added in any step of the above process.

Method 3, it comprises the following steps:
(1) dispersing an active agent, a drug sustained release agent and a drug solubilizer in an excess volatile organic solvent (such as methanol and ethanol), optionally adding a drug solvent, an efficacy enhancer, an antioxidant and an acid-base regulator, and mixing well to obtain a dispersion;
(2) removing the excess volatile organic solvent by, for example, evaporation and / or vacuum pump drying;
(3) optionally, supplementing the drug solvent to the prescription proportion;
(4) optionally, adding the efficacy enhancer and the acid-base regulator in prescription amounts, mixing them well, and filtering the resultant to obtain the non-aqueous sustained release drug delivery system.

In the above method 3, the efficacy enhancer and the acid-base regulator can be added simultaneously or successively with the active agent, the drug solvent, the drug sustained release agent and the drug solubilizer in step (1), or they are not added in step (1), but added in step (4). The above steps (3) and (4) can be carried out simultaneously or successively. The sequence number of these steps is only to distinguish the added substances, and does not represent the proceeding sequence of the steps.

According to practical needs, the above methods can also comprise steps of subpackaging and optional degermation or sterilization. The degermation is, for example, filtration degermation. The sterilizing is, for example, moist heat sterilization.

The sustained release drug delivery system provided by the invention can be used for injection administration, such as subcutaneous or intradermal or intramuscular injection administration, or direct instillation at the incision, or incision infiltration, or administration at the nerve plexus, or injection at articular cavity, or intraocular administration, preferably subcutaneous injection administration, and can also be in other administration forms for external administration.

According to some embodiments, the release duration of the active agent in the drug delivery system can reach at least 12 h. According to some embodiments, the release duration of the active agent in the drug delivery system can reach at least 24 h. According to some embodiments, the release duration of the active agent in the drug delivery system can reach at least 48 h. According to some embodiments, the release duration of the active agent in the drug delivery system can reach at least 72 h.

In some embodiments, the composition has a pain relief time significantly superior to the compositions containing no efficacy enhancer and to the compositions with an amount of the efficacy enhancer beyond the range of the present invention.

The expected total daily dose of the sustained release drug delivery system provided by the invention is 1-1000 mg, preferably 5-500 mg of the active drug.

### Terminology Explanation

The term “conventional sustained release preparation” can be understood as a composition having a similar prescription to the present invention without the efficacy enhancer.

The term “active agent” refers to any compound that is capable of producing beneficial or useful effects or a mixture thereof, and can be understood as a drug.

The term “effective treatment time” can be understood as the length of time that the drug is effective, and can be understood as the duration of action.

The term “effective therapeutic dose” refers to the drug dose required to reach the unit effective treatment time.

The term “sustained release drug delivery system” can be understood as a pharmaceutical composition, a pharmaceutical prescription, a pharmaceutical recipe, formulation or a pharmaceutical preparation.

The term “bioavailability” refers to the speed and degree of the drug being absorbed into human body’s circulation. In the present invention, it is compared by using the area under the plasma concentration vs. time curve (AUC).

The term “effective bioavailability” refers to the area under the plasma concentration vs. time curve that is greater than the minimum effective concentration (MEC).

The non-aqueous sustained release drug delivery system provided by the invention not only can provide improved sustained release effect, improve bioavailability, enhance curative effect, reduce drug dose under the same efficacy, but also can improve effective bioavailability, and reduce the risk of toxic reaction caused by the accumulation of ineffective drugs. The non-aqueous sustained release drug delivery system can controllably adjust the in vivo release rate of the active agent, providing beneficial value for the on-demand selection during drug development. The drug delivery system obtained has good in vivo safety, controllable sustained release effect and high bioavailability, and thus it has a good application prospect.

The non-aqueous sustained release drug delivery system provided by the invention has the following advantages:
1. Compared with the ordinary injection at the same dose, the present invention can significantly reduce the peak plasma concentration and improve the safety in clinical medication.
2. The effective treatment time of the drug delivery system of the invention can be 12 h or more, preferably 24 h or more, which reduces the number of repeated administration, increases the compliance of patients, and reduces the treatment cost.
3. At the same dose, the drug delivery system of the invention can prolong the effective treatment time by at least 25% compared with conventional sustained release preparations, and thus increase the tolerance of patients.
4. Compared with conventional sustained release preparations, the drug delivery system of the invention can significantly improve the bioavailability and effective bioavailability, and reduce the effective therapeutic dose by at least 20%, and thus it can reduce the drug dose under the same efficacy, improve the drug utilization, reduce waste of the drug, avoid the long-term retention of ineffective drugs, and reduce the toxicity risk caused by drug accumulation.
5. The sustained release drug delivery system of the invention has controllable sustained release time, providing the beneficial value for on-demand selection in drug development.
6. Compared with conventional sustained release preparations, the sustained release drug delivery system of the invention has less irritation at the injection site and shows good biocompatibility and safety.

Compared with the developed preparations, the invention has the advantages of good safety, high bioavailability, controllable sustained release effect and high drug utilization.

### BRIEF DESCRIPTION OF THE DRAWINGS

FIG. 1 shows the in vitro release results of a verapamil composition and its aqueous solution.

FIG. 2 shows the in vitro release results of a propranolol composition and its aqueous solution.

FIG. 3 shows the in vitro release results of a vincristine composition and its aqueous solution.

FIG. 4 shows the comparison results of the efficacies of placebo, Exparel and the bupivacaine hydrochloride pharmaceutical compositions (Composition 11 and Composition 16). Compared with the mechanical threshold before administration of each group of animals, ***P < 0.001, **p < 0.01, *p < 0.05; compared with the mechanical threshold of Exparel, ###p < 0.001, ##p < 0.01, #p < 0.05.

FIG. 5 shows the comparison results of the efficacies of placebo, Comparative example 4 and the lidocaine hydrochloride pharmaceutical composition (Composition 1). Compared with the mechanical threshold before administration of each group of animals, ***P < 0.001, **p < 0.01, *p < 0.05; compared with the mechanical threshold of Comparative example 4, ###p < 0.001, ##p < 0.01, #p < 0.05.

FIG. 6 shows the comparison results of the efficacies of placebo, Comparative example 7 and the procaine pharmaceutical composition (Composition 12). Compared with the mechanical threshold before administration of each group of animals, ***P < 0.001, **p < 0.01, *p < 0.05; compared with the mechanical threshold of Comparative Example 7, ###p < 0.001, ##p < 0.01, #p < 0.05.

FIG. 7 shows the in vivo pharmacokinetic (PK) results of the bupivacaine hydrochloride pharmaceutical compositions (Compositions 11 and 16) of the present invention, Comparative example 1 and Exparel.

FIG. 8 shows the in vivo pharmacokinetic (PK) results of the lidocaine hydrochloride pharmaceutical compositions (Compositions 1 and 17) of the present invention, Comparative examples 2 and 4.

FIG. 9 shows the in vivo pharmacokinetic (PK) results of the mepivacaine hydrochloride pharmaceutical composition (Composition 8) of the present invention and Comparative examples 3 and 5.

FIG. 10 shows the histopathological results of the non-injection group, Comparative examples 2 and 4 and the lidocaine hydrochloride pharmaceutical composition (Composition 1) of the present invention at the injection sites.

### DETAILED DESCRIPTION OF EMBODIMENTS

The composition, preparation method and use of the invention are further described through the following preparation examples and experimental examples, but they are not intended to limit the invention. The invention is further described in details below with reference to the examples, but those skilled in the art should understand that the invention is not limited to these examples and the preparation methods used. Moreover, those skilled in the art can make equivalent replacements, combinations, improvements or modifications to the invention according to the description of the invention, and all of these are to be included in the scope of the invention.

### Preparative Examples

### Example 1

300 mg of lidocaine hydrochloride, 1.0 g of benzyl alcohol, 2.0 g of soybean lecithin S100 and 6.2 g of ethyl oleate were precisely weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 0.5 g of fish oil 3322 and mixed well to obtain a liquid, which was filtered, repacked, filled with nitrogen, sealed, and then sterilized through moist heat sterilization to obtain Composition 1.

### Example 2

200 mg of verapamil hydrochloride, 1.0 g of egg yolk lecithin E80, 8.1 g of soybean oil and 0.2 g of linseed oil were precisely weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 0.5 g of anhydrous ethanol and mixed well to obtain Composition 2.

### Example 3

500 mg of promethazine free base, 2.0 g of benzyl alcohol, 2.5 g of egg yolk lecithin E80 and 4.0 g of castor oil were precisely weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 1.0 g of fish oil 3322 and mixed well to obtain Composition 3.

### Example 4

600 mg of huperzine A free base, 6.0 g of benzyl alcohol, 6.0 g of egg yolk lecithin E80 and 16.5 g of ethyl oleate were precisely weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 0.9 g of perilla seed oil and mixed well to obtain Composition 4.

### Example 5

0.6 g of fluvistrom free base was precisely weighed and dissolved in 2.0 g of benzyl alcohol and 1.5 g of benzyl benzoate to obtain a drug solution. 1.0 g of polyoxyl 15 hydroxystearate was weighed and added to 4.5 g of soybean oil with stirring to obtain a clear solution, which was added to the drug solution with stirring. Then 0.4 g of linseed oil was added thereto and stirred well to obtain a liquid, which was filtered, repacked, filled with nitrogen, sealed, and then sterilized through moist heat sterilization to obtain Composition 5.

### Example 6

300 mg of donepezil hydrochloride was precisely weighed and dissolved in a mixed liquid of 1.2 g of benzyl alcohol and 1.8 g of diethylene glycol monoethyl ether to obtain a drug solution. 1.0 g of polyoxyethylene hydrogenated castor oil was added to 5.2 g of castor oil and stirred well. The resultant was added to the drug solution and stirred well. 0.5 g of fish oil 3322 was added thereto and stirred well to obtain a liquid, which was filtered, repacked, filled with nitrogen, sealed, and then sterilized through moist heat sterilization to obtain Composition 6.

### Example 7

Based on the weight percentage of the total weight of the composition, 1% propranolol free base was weighed and dissolved in 18% anhydrous ethanol. 50% soybean lecithin S100 was added thereto and dissolved under stirring at 60° C. 26% castor oil and 5% fish oil 3322 were added and stirred well to obtain a liquid, which was filtered, repacked, filled with nitrogen, and sealed to obtain Composition 7.

### Example 8

Based on the weight percentage of the total weight of the composition, 3% mepivacaine hydrochloride, 40% soybean oil, 40% egg yolk lecithin PC-98T and 2% dexamethasone were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 15% anhydrous ethanol and mixed thoroughly to obtain a liquid, which was filtered, repacked, filled with nitrogen, sealed to obtain Composition 8.

### Example 9

Based on the weight percentage of the total weight of the composition, 1% vincristine free base, 15% benzyl alcohol, 31% medium chain triglycerides, 45% egg yolk lecithin E80 and 8% fish oil 3322 were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. After the flask was cooled to room temperature, Composition 9 was obtained.

### Example 10

Based on the weight percentage of the total weight of the composition, 2% clopidogrel free base, 23% benzyl alcohol, 10% sesame oil, 63% egg yolk lecithin E80 and 2% perilla seed oil were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. After the flask was cooled to room temperature, Composition 10 was obtained.

### Example 11

Based on the weight percentage of the total weight of the composition, 3% bupivacaine hydrochloride was precisely weighed and dissolved in 12% benzyl alcohol, and 30% egg yolk lecithin E80 was added thereto and dissolved with stirring at 60° C., and then 47% castor oil and 8% fish oil 3322 were added and stirred well to obtain a liquid, which was filtered, repacked, filled with nitrogen, sealed and then sterilized through moist heat sterilization to obtain Composition 11.

### Example 12

Based on the weight percentage of the total weight of the composition, 5% procaine free base was precisely weighed and dissolved in 25% benzyl alcohol to obtain a drug solution. 50% egg yolk lecithin PC-98T was weighed and added to 16% medium chain triglyceride, and dissolved with stirring at 60° C. The resultant was added to the drug solution with stirring, and then 4% perilla seed oil was added thereto and stirred well to obtain a liquid, which was filtered, repacked, filled with nitrogen, sealed and then sterilized through moist heat sterilization to obtain Composition 12.

### Example 13

Based on the weight percentage of the total weight of the composition, 3% simvastatin free base, 8% benzyl benzoate, 10% benzyl alcohol, 53% soybean oil, 20% egg yolk lecithin E80 and 6% fish oil 3322 were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. After the flask was cooled to room temperature, Composition 13 was obtained.

### Example 14

Based on the weight percentage of the total weight of the composition, 2% tetracaine free base, 12% benzyl alcohol, 54% castor oil, 27% egg yolk lecithin PC-98T and 5% perilla seed oil were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The obtained liquid was filtered, repacked, filled with nitrogen, sealed and then sterilized through moist heat sterilization to obtain Composition 14.

### Example 15

Based on the weight percentage of the total weight of the composition, 4% nimodipine free base, 40% sesame oil, 34% egg yolk lecithin E80 and 7% linseed oil were weighed into a pre-weighed round bottom flask and added with excess methanol to be completely dissolved by ultrasound. The round bottom flask was connected to a suitable rotary evaporator, and evaporated under reduced pressure until the weight change of the round bottom flask indicated that the methanol had been completely removed. The flask was cooled to room temperature, supplemented with 15% anhydrous ethanol and mixed well to obtain Composition 15.

### Example 16

Based on the weight percentage of the total weight of the composition, 3% bupivacaine hydrochloride, 20% propylene glycol, 31.7% castor oil, 40% soybean lecithin S100 and 0.3% meglumine were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. 5% fish oil 3322 was added and stirred well to obtain a liquid, which was filtered, repacked, filled with nitrogen, sealed and then sterilized through moist heat sterilization to obtain Composition 16.

### Example 17

Based on the weight percentage of the total weight of the composition, 3% lidocaine hydrochloride, 0.1% α-tocopherol, 0.1% ascorbyl palmitate were dissolved a mixed solvent of 10% benzyl alcohol and 5% propylene glycol, and then 35% soybean lecithin S100 was added and dissolved with stirring. Then 40.8% castor oil and 5% fish oil 3322 were added and stirred well, and then added with 1% ethylenediamine and mixed well to obtain a liquid, which was filtered, repacked, filled with nitrogen, sealed and then sterilized through moist heat sterilization to obtain Composition 17.

### Example 18

Based on the weight percentage of the total weight of the composition, 3% mepivacaine hydrochloride, 20% propylene glycol, 33.92% castor oil, 0.08% α-tocopherol, 38% soybean lecithin S100 and 4% linseed oil were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. 1% diethanolamine was added and mixed well to obtain a liquid, which was filtered, repacked, filled with nitrogen, sealed and then sterilized through moist heat sterilization to obtain Composition 18.

### Example 19

Based on the weight percentage of the total weight of the composition, 3% bupivacaine hydrochloride, 20% propylene glycol, 31.7% castor oil, 40% soybean lecithin S100 and 0.3% meglumine were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. 5% fish oil 1812 was added and stirred well to obtain a liquid, which was filtered, repacked, filled with nitrogen, sealed and then sterilized through moist heat sterilization to obtain Composition 19.

### Example 20

Based on the weight percentage of the total weight of the composition, 3% bupivacaine hydrochloride, 20% propylene glycol, 31.7% castor oil, 40% soybean lecithin S100 and 0.3% meglumine were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. 5% algal oil was added and stirred well to obtain a liquid, which was filtered, repacked, filled with nitrogen, sealed and then sterilized through moist heat sterilization to obtain Composition 20.

### Comparative Example 1: Bupivacaine Hydrochloride Injection

150mg of bupivacaine hydrochloride as the active pharmaceutical ingredient (calculated by bupivacaine hydrochloride) was weighed into a vial, added with 20 ml purified water, vortexed and ultrasonicated. After the mixture was dissolved and became clear to obtain a uniform solution, sodium chloride was added to adjust the osmotic pressure to isotonic to obtain a bupivacaine hydrochloride injection.

### Comparative Example 2: Lidocaine Hydrochloride Injection

200 mg of lidocaine hydrochloride as the active pharmaceutical ingredient (calculated by lidocaine hydrochloride) was weighed into a vial, added with 10 ml purified water, vortexed and ultrasonicated. After the mixture was dissolved and became clear, sodium chloride was added to adjust the osmotic pressure to isotonic to obtain a lidocaine hydrochloride injection.

### Comparative Example 3: Mepivacaine Hydrochloride Injection

500 mg of mepivacaine hydrochloride as the active pharmaceutical ingredient (calculated by mepivacaine hydrochloride), 330 mg of sodium chloride, 15 mg of potassium chloride and 16.5 mg of calcium chloride were weighed into a vial, added with 50 ml purified water, vortexed and ultrasonicated. After the mixture was dissolved and became clear, a uniform solution was obtained, which was the mepivacaine hydrochloride injection.

### Comparative Example 4: Lidocaine Hydrochloride Conventional Sustained Release Preparation

300 mg of lidocaine hydrochloride, 1.0 g of benzyl alcohol, 2.0 g of soybean lecithin and 6.7 g of ethyl oleate were precisely weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The obtained liquid was filtered, repacked, filled with nitrogen, sealed and then sterilized through moist heat sterilization to obtain the lidocaine hydrochloride conventional sustained release preparation.

### Comparative Example 5: Mepivacaine Hydrochloride Conventional Sustained Release Preparation

Based on the weight percentage of the total weight of the composition, 3% mepivacaine hydrochloride, 42% soybean oil and 40% egg yolk lecithin were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The flask was cooled to room temperature, added with 15% anhydrous ethanol and mixed well to obtain a liquid, which was filtered, repacked, filled with nitrogen, sealed to obtain the mepivacaine hydrochloride conventional sustained release preparation.

### Comparative Example 6: Procaine Hydrochloride Injection

200 mg of procaine hydrochloride as the active pharmaceutical ingredient (calculated by procaine hydrochloride) was weighed into a vial, added with 20 ml purified water, vortexed and ultrasonicated. After the mixture was dissolved and became clear, sodium chloride was added to adjust the osmotic pressure to isotonic to obtain the procaine hydrochloride injection.

### Comparative Example 7: Procaine Conventional Sustained Release Preparation

Based on the weight percentage of the total weight of the composition, 5% procaine free base was precisely weighed and dissolved in 25% benzyl alcohol to obtain a drug solution. 50% egg yolk lecithin was added to 20% medium chain triglyceride and dissolved with stirring at 60° C., and the resultant was added to the drug solution and stirred well to obtain a liquid, which was filtered, repacked, filled with nitrogen, sealed and then sterilized through moist heat sterilization to obtain the procaine conventional sustained release preparation.

### Comparative Example 8: Bupivacaine Hydrochloride Conventional Sustained Release Preparation

Based on the weight percentage of the total weight of the composition, 3% bupivacaine hydrochloride, 20% propylene glycol, 36.7% castor oil, 40% soybean lecithin S100 and 0.3% meglumine were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. The obtained liquid was filtered, repacked, filled with nitrogen, sealed and then sterilized through moist heat sterilization to obtain the Comparative example 8.

### Comparative Example 9: Bupivacaine Hydrochloride Sustained Release Preparation

Based on the weight percentage of the total weight of the composition, 3% bupivacaine hydrochloride, 20% propylene glycol, 18.7% castor oil, 40% soybean lecithin S100 and 0.3% meglumine were weighed into a pre-weighed round bottom flask and added with excess anhydrous ethanol to be completely dissolved by ultrasound. The round bottom flask was connected to a suitable rotary evaporator, and evaporated under reduced pressure until the weight change of the round bottom flask indicated that the anhydrous ethanol had been completely removed. 18% fish oil 3322 was added and stirred well to obtain a liquid, which was filtered, repacked, filled with nitrogen, sealed and then sterilized through moist heat sterilization to obtain the Comparative example 9.

### Experimental Examples

### Experimental Example 1

### Study on the Stability

The pharmaceutical compositions of the invention were placed under accelerated conditions (temperature (40±2) °C, relative humidity (75±5)%) and long-term conditions (temperature (25±2) °C, relative humidity (60±5)%), and the stability of the compositions were studied by their appearance. The results are shown in Table 1.

Table 1

Study results of the stability of the compositions

| Composition | Appearance | | |
|---|---|---|---|
| | Day 0 | 6 months under accelerated conditions | 6 months under long term conditions |
| Composition 1 | clear and transparent | clear and transparent | clear and transparent |
| Composition 8 | clear and transparent | clear and transparent | clear and transparent |
| Composition 9 | clear and transparent | clear and transparent | clear and transparent |
| Composition 14 | clear and transparent | clear and transparent | clear and transparent |
| Composition 16 | clear and transparent | clear and transparent | clear and transparent |
| Composition 17 | clear and transparent | clear and transparent | clear and transparent |
| Composition 19 | clear and transparent | clear and transparent | clear and transparent |
| Composition 20 | clear and transparent | clear and transparent | clear and transparent |

The results showed that the prepared compositions were clear and transparent after being placed for 6 months under accelerated and long-term conditions, indicating that their storage stabilities are good.

Some drugs were selected as model drugs to study the pharmacodynamics and pharmacokinetics of the compositions in animals.

### Experimental Example 2

### Study on the In Vitro Release Curve

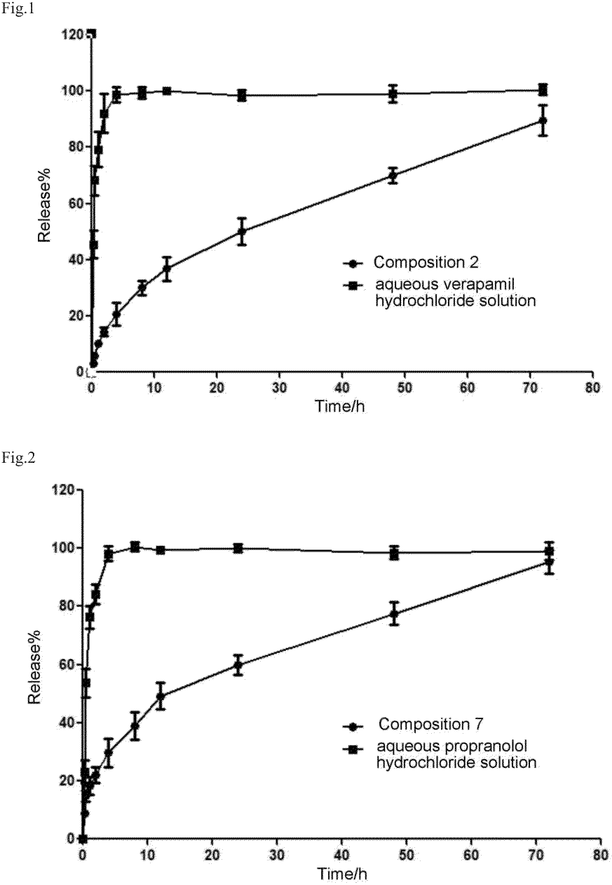

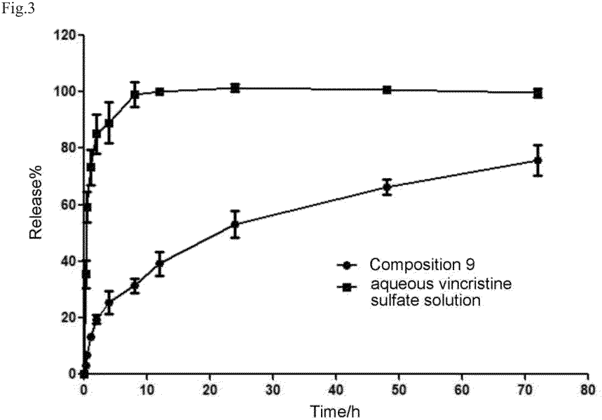

The compositions and their corresponding aqueous solution (see Table 2) were placed respectively in dialysis bags, and immersed in an appropriate amount of phosphate buffers (PBS) at 37° C. 1 ml of release solutions were taken at different time points, and 1 ml of PBS solution was quickly supplemented with until the release was completed. The cumulative release curve was plotted with time as the abscissa and the cumulative release rate as the ordinate. The results are shown in FIGS. 1-3.

Table 2

In vitro release information of the compositions

| | | |
|---|---|---|
| Composition | Comparative example | Preparations of Comparative examples |
| Composition 2 | aqueous Verapamil hydrochloride solution | 20 mg of verapamil hydrochloride was weighed and dissolved in 8ml purified water, and adjusted to isotonic with sodium chloride. |
| Composition 7 | aqueous Propranolol hydrochloride solution | 20 mg of propranolol hydrochloride was weighed and dissolved in 20 ml purified water, and adjusted to isotonic with sodium chloride. |
| Composition 9 | aqueous Vincristine sulfate solution | 10 mg of vincristine sulfate was weighed and dissolved in 10 ml purified water, and adjusted to isotonic with sodium chloride. |

The results showed that it took at least 72 hours to release 80% of the active pharmaceutical ingredient for the composition of the invention, while it took about 2 hours to release more than 80% of the active pharmaceutical ingredient for the corresponding aqueous solution with an obvious burst release, indicating that the composition of the invention can reduce burst release compared with its aqueous solution, and has advantages in sustained release.

### Experimental Example 3

In the following experimental description, the dose refers to the dose calculated based on the amount of the hydrochloride of the active ingredient in the preparations (that is, converted to hydrochloride in the case of using a free base). Exparel is bupivacaine polycystic liposome, batch number: 18-4122, manufacturer: Pacira.

In the following experiments, the effective therapeutic dose R is calculated according to the following formula: R=A/Tₜ

wherein, A is the dosage of the drug, and Tt is the in vivo effective treatment time of the drug (i.e. the duration of action, determined by pharmacodynamic experiments).

### In Vivo Pharmacodynamic Study of Pharmaceutical Compositions (1)

Animals for experiment: male SD rats with a weight of 200-300 g were fed adaptively for 2-4 days, and the basic thresholds were measured every day for 3 days.

Experimental grouping and dosage: the rats were randomly grouped according to the basic thresholds, 6 rats/group. 10 mg/kg comparative example 1, 30 mg/kg composition 11, 20 mg/kg composition 11, 30 mg/kg composition 16, 30 mg/kg Exparel and normal saline placebo were respectively injected into the subcutaneous tissue of rats’ ootpad (the dosage volume range was 0.10-0.35 ml), and the pain threshold was measured by von Frey filament pain meter.

Method: paired t-test was used to compare the mechanical paw withdrawal threshold with that before administration for each group of animals, and unpaired t-test was used to compare the mechanical paw withdrawal threshold with that of Exparel for each group of animals. P < 0.05 means there is a statistical difference, P < 0.01 and P < 0.001 mean there is a significant statistical difference.

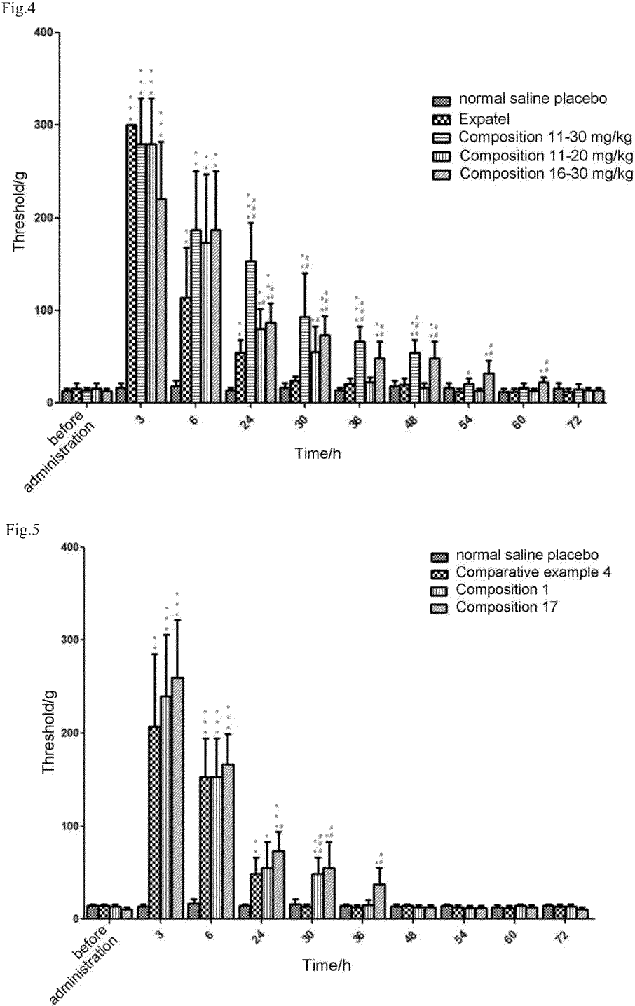

The experimental results are shown in FIG. 4.

Compared with the mechanical threshold before administration for each group, the duration of action of Comparative example 1 was 2 h, the duration of action of the marketed Exparel in rats was about 24 h, while the duration of action of Composition 11 of the invention could reach 48 h at the same dose, the duration of action of Composition 11 which had a reduced dose was 30 h, and the duration of action of Composition 16 was 54 h. It can be seen that the duration of action of the composition of the invention is at least twice longer than that of Exparel at the same dose. Compared with Exparel at the same dose, the pharmacodynamic intensities of Composition 11 at 24-54 h and Composition 16 at 24-60 h had significantly difference, indicating that the duration of action of the composition of the invention has a significant advantage over those of Comparative examples 8 and 9.

The effective therapeutic dose R was calculated, and the results are shown in Table 3.

Table 3

Calculation results of effective therapeutic dose of the compositions and comparative examples

| Composition | R |
|---|---|
| Comparative example 1 | 5.0 |
| Composition 11 (30 mg/kg) | 0.63 |
| Composition 11 (20 mg/kg) | 0.67 |
| Composition 16 (30 mg/kg) | 0.56 |
| Exparel | 1.25 |

It can be seen from the results that the effective therapeutic dose R of the compositions 11 and 16 of the invention were reduced by at least 40% compared with those of Exparel and Comparative example 1, and thus the compositions of the present application have improved drug utilization.

### In Vivo Pharmacodynamic Study of Pharmaceutical Compositions (2)

According to the procedures of the in vivo pharmacodynamic study of pharmaceutical compositions (1), the efficacies of 30 mg/kg Comparative example 8, Comparative example 9, Composition 16, Composition19 and Composition 20 in rats were studied respectively. See Table 4 for the experimental results.

Table 4

Mechanical pain threshold results of the compositions

| Time/h | Comparative example 8 | Comparative example 9 | Composition 16 | Composition 19 | Composition 20 |
|---|---|---|---|---|---|
| Before administration | 12±3 | 13±3 | 13±3 | 13±3 | 13±3 |
| 3 | 240±66*** | 240±66*** | 220±62*** | 280±49*** | 247±86** |
| 6 | 173±73** | 173±73** | 187±64** | 153±41*** | 167±33*** |
| 24 | 80±22*** | 73±21** | 87±21*** | 80±22*** | 80±22*** |
| 30 | 36±19** | 36±19* | 73±21 **##&& | 73±21 **##&& | 49±18** |
| 36 | 13±3 | 13±3 | 49±18**###&&& | 54±14**###&&& | 36±19*#& |
| 48 | 15±6 | 13±3 | 49±18**##&&& | 43±19*##&& | 24±4**#&&& |
| 54 | 15±6 | 12±3 | 32±14*#&& | 24+4**#&&& | 13±3 |
| 60 | 13±3 | 13±3 | 22±6*##&& | 14±2 | 13±3 |
| 72 | 14±2 | 13±3 | 14±2 | 12±3 | 13±3 |

Notes:
*P<0.05, **p<0.01, ***p<0.001 v.s. threshold before administration;
#P<0.05, ##p<0.01, ###p<0.001 v.s. Comparative example 8;
&p<0.05, &&p<0.01, &&&p<0.001 v.s. Comparative Example 9.

The results showed that the duration of action of Comparative examples 8 and 9 and Compositions 16, 19 and 20 in rats were 30 h, 30 h, 60 h, 54 h and 48 h, respectively. It can be seen that at the same dose, the duration of action of Compositions 16, 19 and 20 (both containing 5% efficacy enhancer) can be prolonged by at least 60% compared with that of Comparative example 8 (not containing the efficacy enhancer) and Comparative example 9 (containing 18% efficacy enhancer). Fish oil 3322 contains about 33% of eicosapentaenoic acid and about 22% of docosahexaenoic acid, fish oil 1812 contains about 18% of eicosapentaenoic acid and about 12% of docosahexaenoic acid, and algal oil contains about 35% of docosahexaenoic acid with no eicosapentaenoic acid. With the same ratio of efficacy enhancers, the durations of action of Compositions 16, 19 and 20 were shortened in turn, indicating that the content of eicosapentaenoic acid and docosahexaenoic acid in the composition may affect the duration of action, and the pharmacodynamic result is better in the case that eicosapentaenoic acid and docosahexaenoic acid are both contained.

In addition, compared with Comparative examples 8 and 9, the pain thresholds of Compositions 6, 19 and 20 at 30 h or different time points after 30 h had significantly difference, indicating that the composition of the present invention has a significant advantage in the duration of action over Comparative examples 8 and 9, and the efficacy enhancer has achieved unexpected effects for a certain mechanism.

The effective therapeutic dose of each composition was calculated according to the duration of action of the composition. The results are shown in Table 5.

Table 5

Calculation results of effective therapeutic dose of the compositions

| parameter | Comparative example 8 | Comparative example 9 | Composition 16 | Composition 19 | Composition 20 |
|---|---|---|---|---|---|
| R | 1.0 | 1.0 | 0.5 | 0.56 | 0.63 |

It can be seen that the effective therapeutic dose of the composition of the invention was reduced by at least 30% compared with those of Comparative examples 8 and 9, indicating that the composition of the invention has improved the drug utilization.

### In Vivo Pharmacodynamic Study of Pharmaceutical Compositions (3)

According to the procedures of in vivo pharmacodynamic study of pharmaceutical compositions (1), the efficacy of 10 mg/kg Comparative example 2, 30 mg/kg Comparative example 4, 30 mg/kg Composition 1 and 30 mg/kg Composition 17 in rats were studied respectively. The experimental results are shown in FIG. 5.

Compared with the mechanical threshold before administration for each group, the duration of action of Comparative example 2 was 3 h, and the duration of action of Comparative example 4 could maintain about 24 h, while the Composition 1 and Composition 17 of the invention could reach 30 h and 36 h at the same dose. It can be seen that at the same dose, the duration of action of the compositions of the invention was prolonged by at least 25% compared with the comparative examples. Compared with Comparative example 4 at the same dose, the pharmacodynamic intensities of Composition 1 in 30 h and Composition 17 at 24-36 h were significantly different, indicating that the compositions of the invention have significant advantage in the duration of action over Comparative example 4.

The effective therapeutic dose R was calculated, and the results are shown in Table 6.

Table 6

Calculation results of effective therapeutic dose of the compositions and comparative examples

| Composition | R |
|---|---|
| Comparative example 2 | 3.3 |
| Comparative example 4 | 1.25 |
| Composition 1 | 1.0 |
| Composition 17 | 0.83 |

It can be seen from the results that the effective therapeutic doses R of the compositions 1 and 17 of the invention were reduced by at least about 20% compared with Comparative examples 2 and 4, and thus the compositions of the present application have improved drug utilization.

### In Vivo Pharmacodynamic Study of Pharmaceutical Compositions (4)

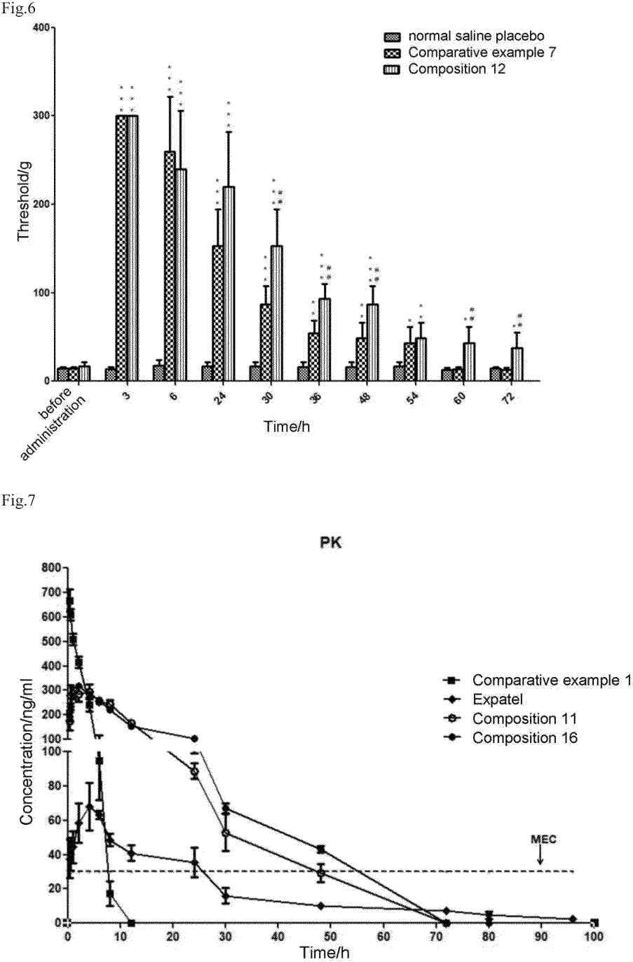

According to the procedures of in vivo pharmacodynamic study of pharmaceutical compositions (1), the efficacy of 20 mg/kg Comparative example 6, 60 mg/kg Comparative example 7 and 60 mg/kg Composition 12 in rats were studied respectively. The experimental results are shown in FIG. 6.

Compared with the mechanical threshold before administration for each group, the duration of action of Comparative example 6 was 2 h, the duration of action of Comparative example 7 was about 54 h, and the duration of action of Composition 12 of the invention could reach 72 h at the same dose. It can be seen that at the same dose, the duration of action of the compositions of the invention was prolonged by at least 30% compared with comparative examples. Compared with Comparative example 7 at the same dose, the pharmacodynamic intensity of Composition 12 at 60-72 h was significantly different, indicating that the composition of the invention has a significant advantage in the duration of action over Comparative example 7.

The effective therapeutic dose R was calculated, and the results are shown in Table 7.

Table 7

Calculation results of effective therapeutic dose of the composition and comparative examples

| Composition | R |
|---|---|
| Comparative example 6 | 10 |
| Comparative example 7 | 1.11 |
| Composition 12 | 0.83 |

It can be seen from the results that the effective therapeutic dose R of Composition 12 of the invention was reduced by at least about 25% compared with Comparative examples 6 and 7, and the drug utilization is improved.

In conclusion, the pharmaceutical composition of the invention has a controllable sustained release time, and its duration of action is prolonged by at least 25%, and its effective therapeutic dose R is reduced by at least about 20% compared with the conventional sustained release preparation at the same dose, and thus has obvious pharmacodynamic advantages, indicating that the efficacy enhancer has achieved unexpected effects for a certain mechanism.

### Experimental Example 4

In the following experimental description, the dose refers to the dose calculated based on the amount of hydrochloride of the active ingredient in the preparations (that is, converted to hydrochloride in the case of using a free base). Exparel is bupivacaine polycystic liposome, batch number: 18-4122, manufacturer: Pacira.

### In Vivo Pharmacokinetic Study of Pharmaceutical Compositions (1)

The obtained pharmaceutical composition was subcutaneously injected into male SD rats, blood was taken at regular intervals, and the drug concentration in plasma was determined by LC-MS method to study the pharmacokinetics in vivo.

Animals for experiment: male SD rats, weight 200-300 g, source: Shanghai Institute of Materia Medica, Chinese Academy of Sciences.

Experimental grouping: rats were randomly grouped according to the following table, 6 rats/group.

See Table 8 for the preparations and doses.

Table 8

Dose information of the compositions

| Composition | Dose/mg/kg |
|---|---|
| Comparative example 1 | 10 |
| Composition 11 | 30 |
| Exparel | 30 |
| Composition 16 | 30 |

The pharmacokinetic (PK) parameters of the compositions were calculated, and the results were statistically analyzed by t-test. The experimental results are shown in Table 9 and FIG. 7.

Table 9

PK parameters of the compositions

| PK parameters | Comparative example 1 | Exparel | Composition 11 | Composition 16 |
|---|---|---|---|---|
| Cₘₐₓ(ng/ml) | 668.13±44.63 | 68.02±14.01 | 294.42±29.49 | 317.00±12.12 |
| AUC(h*ng/ml) | 2125.27±55.81 | 1758.31±202.26 | 6005.23±201.69*** | 6452.08±177.50*** |
| AUC_{C>MEC}(h*ng/ml) | / | 1123.96±92.31 | 5663.61±209.84*** | 6158.83±165.68*** |

Note: AUC_{C >} _{MEC} is the effective bioavailability.
Note: *p<0.05 ** p<0.01 *** P < 0.001 v.s. Exparel.

The results showed that the Cₘₐₓ of Compositions 11 and 16 was reduced by at least 80% compared with that of Comparative example 1. Compared with Exparel, the bioavailability and effective bioavailability of Compositions 11 and 16 were significantly improved.

### In Vivo Pharmacokinetic Study of Pharmaceutical Compositions (2)

The pharmacokinetic study was carried out according to the procedures of in vivo pharmacokinetic study of pharmaceutical compositions (1).

See table 10 for the preparations and doses.

Table 10

Dose information of the compositions

| Drug | composition | Dose/mg/kg |
|---|---|---|
| Lidocaine hydrochloride | Comparative example 2 | 10 |
| | Comparative example 4 | 30 |
| | Composition 1 | 30 |
| | Composition 17 | 30 |

The pharmacokinetic (PK) parameters of the compositions were calculated, and the results were statistically analyzed by t-test. The experimental results are shown in Table 11 and

Table 11

PK parameters of the pharmaceutical compositions

| PK parameters | Comparative example 2 | Comparative example 4 | Composition 1 | Composition 17 |
|---|---|---|---|---|
| Cₘₐₓ(ng/ml) | 292.18±43.04 | 197.32±30.21 | 214.56±30.21 | 236.67±12.06 |
| AUC(h*ng/ml) | 1294.78±101.23 | 2727.89±156.92 | 3490.60±273.45*** | 3974.48±28.86*** |
| AUC_{C>MEC}(h*ng/ml) | / | 2108.64±102.12 | 3225.01±154.62*** | 3817.01±11.65*** |

Note: AUC_{C >} _{MEC} is the effective bioavailability.
Note: *p<0.05 ** p<0.01 *** P < 0.001 v.s. Comparative example 4.

The results showed that the Cₘₐₓ of Compositions 1 and 17 was reduced by at least 70% compared with that of Comparative example 2. Compared with that of Comparative example 4, the bioavailability and effective bioavailability of Compositions 1 and 17 were significantly improved.

### In Vivo Pharmacokinetic Study of Pharmaceutical Compositions (3)

The pharmacokinetic study was carried out according to the procedures of in vivo ph armacokinetic study of pharmaceutical compositions (1).

See table 12 for the preparations and doses.

Table 12

Dose information of the compositions

| Drug | composition | Dose/mg/kg |
|---|---|---|
| Mepivacaine hydrochloride | Comparative example 3 | 7 |
| | Comparative example 5 | 21 |
| | Composition 8 | 21 |

The pharmacokinetic (PK) parameters of the compositions were calculated, and the results were statistically analyzed by t-test. The experimental results are shown in Table 13 and FIG. 9.

Table 13

PK parameters of the pharmaceutical compositions

| PK parameters | Comparative example 3 | Comparative example 5 | Composition 8 |
|---|---|---|---|
| Cₘₐₓ(ng/ml) | 359.38±39.02 | 155.09+23.12 | 126.23±11.44 |
| AUC(h*ng/ml) | 1474.87±103.76 | 3101.36+239.87 | 3880.72±221.09*** |
| AUC_{C>MEC}(h*ng/ml) | / | 2667.81±123.76 | 3826.65±145.73*** |

Note: AUC_{C >} _{MEC} is the effective bioavailability.
Note: *p<0.05 ** p<0.01 *** P < 0.001 v.s. comparative example 5.

The results showed that the Cₘₐₓ of Composition 8 was reduced by 88% compared with that of Comparative example 3, and the bioavailability and effective bioavailability of Composition 8 were significantly improved compared with that of Comparative example 5.

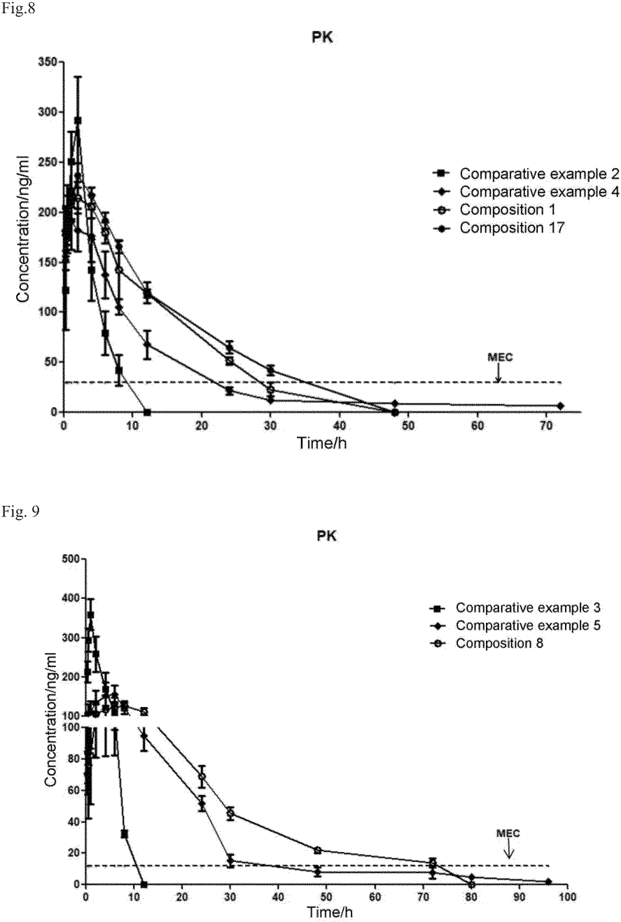

The results of FIGS. 7-9 showed that in the comparative examples, the drug stayed in the body for a long time at a low concentration after the effective treatment time was reached, while compared with the conventional sustained release preparation of comparative examples, the composition of the invention could not only prolong the effective treatment time, but also reduce the retention time of the drug at an invalid concentration in the body and reduce the toxic reaction caused by drug accumulation.

Conclusion: Composition 11 vs Comparative example 1, Composition 1 vs Comparative example 2 and Composition 8 vs Comparative example 3 have a Cₘₐₓ reduced by at least 70% respectively with improved safety. Comparing the compositions of the invention with the marketed sustained release preparation or conventional sustained release preparation, the bioavailability and effective bioavailability are significantly improved, the retention time of the drug at an invalid concentration in the body is decreased, and the risk of adverse reactions caused by drug accumulation is reduced.

### Experimental Example 5

### Study on the Local Irritation of Pharmaceutical Compositions

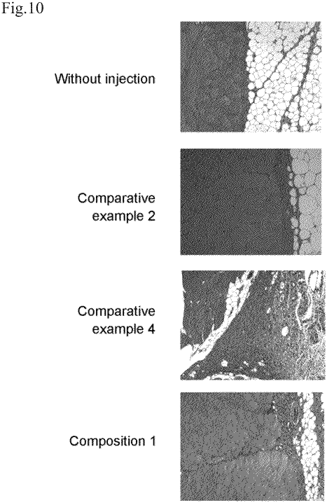

In order to further study the irritation of the non-aqueous sustained release drug delivery system of the invention at the injection site, 10 mg/kg Comparative example 2, 30 mg/kg Comparative example 4 and 30 mg/kg Composition 1 were subcutaneously injected into rats, and after 48 hours, the tissue at the injection site was taken for morphological observation. The results are shown in FIG. 10.

FIG. 10 showed that when the composition or the comparative examples were injected into the body, there would be a certain degree of irritant response, which is an immune response of the body. Inflammatory cell infiltration appeared in the group of Comparative example 4 with serious irritant reaction. The irritation reaction was significantly reduced in the group of Composition 1, indicating that the drug delivery system of the invention has better local tolerance than conventional sustained release preparations. Moreover, no obvious abnormality was observed visually at the injection site of rats in the group of Composition 1, reflecting that the composition of the invention has good safety.

To sum up, the drug delivery system of the invention can significantly reduce Cₘₐₓ and improve the drug safety compared with the common injection at the same dose. At the same dose, compared with the conventional sustained release preparation, the drug delivery system of the invention can prolong the duration of action by at least 25%, and thus has obvious pharmacodynamic advantages. The drug delivery system of the invention can reduce the effective therapeutic dose R per unit effective treatment time by at least about 20%. Compared with the marketed sustained release preparation or conventional sustained release preparation, the composition of the invention has significantly improved bioavailability and effective bioavailability, can avoid the long-term retention of the drug at an invalid concentration in the body and reduce the risk caused by drug accumulation. Compared with the studied preparation, the invention has the advantages of good safety, high bioavailability, controllable sustained release effect and high drug utilization.

1. A non-aqueous sustained release drug delivery system, comprising: an active agent, a drug solvent, a drug sustained release agent, a drug solubilizer, an efficacy enhancer, an optional antioxidant, and an optional acid-base regulator, wherein,
the drug solvent is one or more selected from the group consisting of benzyl alcohol, ethanol, glycerol, isopropanol, liquid polyethylene glycol, polyethylene glycol monomethyl ether, glycerol monoacetate, diethylene glycol monoethyl ether, ethyl lactate, tetrahydrofuran polyethylene glycol ether, benzyl benzoate, dimethyl acetamide, N-methyl pyrrolidone, 2-pyrrolidone, propylene glycol, methyl acetate, ethyl acetate, propylene glycol diacetate, diethyl malonate, glyceryl triacetate, dimethylformamide, dimethyl sulfoxide, caprolactam, triethyl citrate, and propylene carbonate,
the drug sustained release agent is one or more selected from the group consisting of biodegradable polymers and pharmaceutical oils,
the drug solubilizer is one or more selected from the group consisting of pharmaceutical surfactants,
the efficacy enhancer is one or more selected from the group consisting of ω-3 fatty acids and metabolites thereof, substances rich in ω-3 fatty acids or metabolites thereof, glucocorticoids, and phosphodiesterase-4 inhibitors,
the antioxidant is one or more selected from the group consisting of cysteine, α-tocopherol, α-tocopherol acetate, N-acetyl-L-cysteine, butylated hydroxyanisole, dibutylated hydroxytoluene, propyl gallate, tert-butyl hydroquinone, lipoic acid, tea polyphenols, L-ascorbyl palmitate, glutathione, and
the acid-base regulator is one or more selected from the group consisting of arginine, lysine, histidine, glycine, tromethamine, diethanolamine, ethylenediamine, meglumine, hydrochloric acid, acetic acid, anhydrous citric acid, ascorbic acid, lactic acid, tartaric acid, methanesulfonic acid, methionine, sodium hydroxide, and triethanolamine.

2. The non-aqueous sustained release drug delivery system according to claim 1, comprising:
based on the total weight of the sustained release drug delivery system, in weight percentage,
0.1% to 20% of the active agent;
0.5% to 50% of the drug solvent;
1% to 98% of the drug sustained release agent;
1% to 85% of the drug solubilizer;
0.1% to 10% of the efficacy enhancer;
0% to 1% of the optional antioxidant; and
0% to 8% of the optional acid-base regulator.

3. The non-aqueous sustained release drug delivery system according to claim 1 wherein, the active agent includes at least one of: anticancer drugs, anti-inflammatory drugs, anti-infective drugs, painkillers, hormones, anti-diabetic drugs, anti-hypertension drugs, anti-AIDS drugs, immune enhancers, anti-viral drugs, cardiotonics, anti-obesity drugs, bone metabolism regulators, antiepileptics, anticonvulsants, antidepressants, antipsychotics, anti-Parkinson’s disease drugs, urinary tract drugs, contraceptives, anti-osteoporosis drugs, protein assimilation agents, smoking cessation aids, and cell adhesion promoters.

4. The non-aqueous sustained release drug delivery system according to claim 1, wherein the drug sustained release agent is one or more selected from the group consisting of pharmaceutical oils.

5. The non-aqueous sustained release drug delivery system according to claim 1, wherein,
the biodegradable polymer is one or more selected from the group consisting polylactic acid (PLA), polylactic acid-glycolic acid copolymer (PLGA), polyorthoesters, sucrose acetate iso-butyrate, glycerin fatty acid ester, PEGylated PLA/PLGA, PLGA-PEG-PLGA copolymer, triethylene glycol poly(orthoester) polymer, chitosan, water-soluble carboxymethyl chitosan, fibroin, poly-β-hydroxybutyrate valerate, polylactide/lactide-polyethylene glycol copolymer, polylactide/lactide-polyethylene glycol blend, polycaprolactone-polyethylene glycol copolymer, blend of poly β-hydroxybutyrate and polyethylene glycol, and blend of polylactic acid/hydroxyacetic acid,
the pharmaceutical oil is one or more selected from the group consisting of castor oil, sesame oil, soybean oil, sunflower seed oil, peanut oil, corn oil, rapeseed oil, olive oil, cotton seed oil or other natural vegetable oils, semi-natural oils artificially modified from natural vegetable oils, purified oils and corresponding derivatives; and synthetic oils, including medium chain triglycerides with a carbon chain length of C₆-C₁₂, long chain triglycerides with a carbon chain length of C₁₃-C₂₄, triacetin or other corresponding derivatives, and ethyl oleate,
the pharmaceutical surfactant is one or more selected from the group consisting of pharmaceutical phospholipids, polyoxyl 15 hydroxystearate, polysorbate, polyoxyethylene castor oil, poloxamer, polyoxyethylene fatty acid ester, phosphatidylcholine phosphatidylglycerol, polyethylene glycol, polyethylene glycol monomethyl ether, gelatin,
the ω-3 fatty acids and metabolites thereof is one or more selected from the group consisting of ω-3 polyunsaturated fatty acids and metabolites thereof,
the substances rich in ω-3 fatty acids or metabolites thereof is one or more selected from the group consisting of substances rich in α-linolenic acid, substances rich in α-linolenic acid metabolites,
the glucocorticoids is one or more selected from the group consisting of prednisone, methylprednisone, betamethasone, beclomethasone propionate, prednisolone, hydrocortisone, dexamethasone, and combinations thereof, and
the phosphodiesterase-4 inhibitors is one or more selected from the group consisting of roflumilast, rolipram, penntoxifylline, and combinations thereof.

6. The non-aqueous sustained release drug delivery system according to claim 1, wherein,
the drug sustained release agent is one or more selected from the group consisting of castor oil, sesame oil, ethyl oleate, soybean oil, medium chain triglyceride, and peanut oil, the pharmaceutical surfactant is one or more selected from the group consisting of pharmaceutical phospholipids,
the ω-3 polyunsaturated fatty acids is one or more selected from the group consisting of α-linolenic acid, eicosapentaenoic acid (EPA), docosahexaenoic acid (DHA), and combinations thereof,
the substance rich in α-linolenic acid is one or more selected from the group consisting of linseed oil, perilla seed oil, walnut oil, argan oil, or combinations thereof,
the substance rich in α-linolenic acid metabolites is one or more selected from the group consisting of fish oil, laver oil, algal oil, and combinations thereof.

7. The non-aqueous sustained release drug delivery system according to claim 1, wherein the substance rich in ω-3 fatty acids or metabolites thereof is one or more selected from the group consisting of substances rich in ω-3 fatty acids or metabolites thereof with eicosapentaenoic acid content of not less than 15% and docosahexaenoic acid content of not less than 10%.

8. The non-aqueous sustained release drug delivery system according to claim 1, wherein the system is administered by injection or by other forms of external administration.

9. The non-aqueous sustained release drug delivery system according to claim 1, wherein the release duration of the active agent in the drug delivery system reaches at least 12 h.

10. The non-aqueous sustained release drug delivery system according to claim 1, wherein a total amount administered per day of the sustained release drug delivery system is 1 to 1000 mg of the active drug.

11. The non-aqueous sustained release drug delivery system according to claim 1, comprising:
based on the total weight of the sustained release drug delivery system, in weight percentage,
0.5% to 15% of the active agent;
2% to 40% of the drug solvent;
5% to 90% of the drug sustained release agent;
5% to 80% of the drug solubilizer;
1% to 10% of the efficacy enhancer;
0% to 0.5% of the antioxidant; and
0% to 5% of the acid-base regulator.

12. The non-aqueous sustained release drug delivery system according to claim 1, comprising:
based on the total weight of the sustained release drug delivery system, in weight percentage,
1% to 10% of the active agent;
5% to 35% of the drug solvent;
10% to 82% of the drug sustained release agent;
10% to 65% of the drug solubilizer;
2% to 8% of the efficacy enhancer;
0% to 0.3% of the antioxidant; and
0% to 2% of the acid-base regulator.

13. The non-aqueous sustained release drug delivery system according to claim 6, wherein,
the drug sustained release agent is one or more selected from the group consisting of castor oil, soybean oil, and sesame oil,
the pharmaceutical phospholipids is one or more selected from the group consisting of natural phospholipids, semi-synthetic phospholipids, and synthetic phospholipids, and
the α-linolenic acid is the α-linolenic acid (ALA) of plant origin.

14. The non-aqueous sustained release drug delivery system according to claim 13, wherein,
the natural phospholipids is one or more selected from the group consisting of egg yolk lecithin, soybean lecithin, and combinations thereof,
the semi-synthetic phospholipids is one or more selected from the group consisting of hydrogenated egg yolk lecithin, hydrogenated soybean phospholipids or combinations thereof, and
the synthetic phospholipids is one or more selected from the group consisting of dipalmitoyl phosphatidylethanolamine, dipalmitoyl phosphatidic acid,
dipalmitoylphosphatidylglycerole, dioleoylphosphatidylethanolamine,
dipalmitoylphosphatidylcholine, distearoylphosphatidylcholine, dimyristoylphosphatidylcholine, and combinations thereof.

15. The non-aqueous sustained release drug delivery system according to claim 7, wherein the substance rich in ω-3 fatty acids or metabolites thereof is one or more selected from the group consisting of substances rich in ω-3 fatty acids or metabolites thereof with eicosapentaenoic acid content of not less than 20% and docosahexaenoic acid content of not less than 10%.

16. The non-aqueous sustained release drug delivery system according to claim 7, wherein the substance rich in ω-3 fatty acids or metabolites thereof is one or more selected from the group consisting of fish oil and laver oil.

17. The non-aqueous sustained release drug delivery system according to claim 8, wherein the injection administration includes at least one of subcutaneous, intradermal, intramuscular injection administration, direct instillation at the incision, incision infiltration administration, administration at the nerve plexus, injection at articular cavity, and intraocular administration.

18. The non-aqueous sustained release drug delivery system according to claim 1, wherein a release duration of the active agent in the drug delivery system reaches at least 24 h.

19. The non-aqueous sustained release drug delivery system according to claim 1, wherein a release duration of the active agent in the drug delivery system reaches at least 48 h.

20. The non-aqueous sustained release drug delivery system according to claim 1, wherein a release duration of the active agent in the drug delivery system reaches at least 72 h.
